(19) United States
(12) Patent Application Publication
(10) Pub. No.: US 2021/0008107 A1
(43) Pub. Date: Jan. 14, 2021

(54) CIML NK cells and Methods Therefor

(71) Applicant: NANTKWEST, INC., San Diego, CA (US)

(72) Inventors: Rohit Duggal, San Diego, CA (US); Ranjeet Sinha, San Diego, CA (US); Wenzhao Li, San Diego, CA (US); Jason Isaacson, San Diego, CA (US); Patrick Soon-Shiong, San Diego, CA (US)

(21) Appl. No.: 16/505,625

(22) Filed: Jul. 8, 2019

(51) Int. Cl. A61K 35/17; C12N 5/0783; C07K 14/54; C07K 14/735

(52) U.S. Cl. CPC A61K 35/17; C12N 5/0638; C07K 14/5434; C07K 2319/30; C12N 2501/2312; C12N 2501/2318; C12N 2501/2315; C07K 14/70535

(57) ABSTRACT

Cytokine induced memory like (CIML) NK cells with enhanced cytotoxicity are presented. Most typically, the CIML NK cells are derived from a mononuclear cell fraction of peripheral blood or cord blood. In further contemplated aspects, the CIML NK cells are expanded and induced in a contained and automated production environment that substantially reduces operational complexity and production cost.

### FIELD OF THE INVENTION

The present disclosure relates to compositions, methods, and devices to generate and/or cultivate activated immune competent cells, especially as it relates memory like NK cells that are produced from cord blood (CB) or peripheral blood (PB).

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Natural killer (NK) cells constitute a group of innate immune cells, which are often characterized as cytotoxic lymphocytes that exhibit antibody dependent cellular toxicity via target-directed release of granulysin and perforin. Most NK cells have a specific cell surface marker profile (e.g., CD3⁻, CD56⁺, CD16⁺, CD57⁺, CD8⁺) in addition to a collection of various activating and inhibitory receptors. While more recently NK cells have become a significant component of certain cancer treatments, generation of significant quantities of NK cells (and especially autologous NK cells) has been a significant obstacle as the fraction of NK cells in whole blood is relatively low.

To obtain therapeutically meaningful quantities of NK and NK-like cells, NK cells can be generated from various precursor cells. For example, various stem cell factors (SCF), FLT3 ligand, interleukin (IL)-2, IL-7 and IL-15 have been reported in various in vitro approaches to induce and expand cord blood-derived cytokine-induced killer (CIK) cells (Anticancer Research 30: 3493-3500 (2010)). Similarly, CD34⁺ hematopoietic cells can be exposed to IL-12 and other agents as is reported in US 2018/0044636. In still other approaches, human hemangioblasts were sequentially exposed to two different cytokine cocktails as described in WO2011/068896, and different cytokine cocktails were used with post-embryonic hematopoietic stem cells as taught in WO2012/128622. While at least some of these methods provide a significant n-fold expansion of NK cells, methods and reagents for such expansion are both time and resource demanding. Still further, it should be noted that many of the known methods also require NK cell culture on a feeder cell layer, which is often problematic from a technical and a regulatory perspective.

In more simplified methods, acute myeloid leukemia (AML) cells can be exposed to TpoR agonists to so induce the AML cells to form NK cells. However, such approach is likely not viable as a source for therapeutic cell preparations. Alternative methods have also relied on culturing peripheral blood cells in the presence of various interleukins, stem cell factors, and FLT3 ligands as is disclosed in WO 2011/103882. In yet another method, US 2013/0295671 teaches methods of stimulating already existing NK cells with anti-CD16 and anti-CD3 antibodies along with cytokines. While procedurally simpler, such methods still require elaborate manipulation of the cells and add significant costs due to the specific reagent required.

In still further known methods, U.S. Pat. No. 10,125,351 describes use of cord blood or peripheral blood as a source of cells that are subject to density gradient separation to isolate nucleated cells that are then cultivated with a medium that contains interferon, interleukin, a CD3 antibody and human albumin. Most advantageously, such method is amenable to perfusion culture in a bioreactor and as such significantly reduces operational difficulties. Unfortunately, however, the yield of NK cells is relatively low.

Regardless of the specific manner of production, cultivated NK cells will typically not exhibit memory like character, which is particularly desirable for cancer immune therapy. In at least some attempts to produce memory like NK cells, cultivated NK cells were exposed to IL-12, IL-15, and IL-18 and so exposed NK cells exhibited a memory like phenotype and correlated with the expression of CD94, NKG2A, NKG2C, and CD69 and a lack of CD57 and KIR (see Blood (2012) Vol. 120, No. 24; 4751-4760). Similarly, memory like NK cells were prepared by pre-activating NK cells using various stimulatory cytokines followed by contacting the pre-activated cells with PM21 particles, EX21 exosomes, or FC21 feeder cells as is described in WO 2018/089476. In yet another approach to generate memory like NK cells, freshly isolated NK cells were exposed to an IL-18/IL-12-TxM fusion protein complex as is described in WO 2018/165208. While such methods typically produced at least some NK cells with memory-like character, the cytotoxicity of such activated NK cells against selected target cells was still less than optimal, possibly due to lack or low expression of specific activating receptors and/or expression of specific inhibitory receptors.

Thus, even though various methods of generating memory like NK cells are known in the art, all or almost all of them suffer from various disadvantages. Consequently, there is a need to provide improved systems and methods that produce memory like NK cells, and especially autologous memory like NK cells in significant quantities. Moreover, improved systems and methods will also allow for automation of cell culture and NK cell activation and will have substantially reduced reagent requirements to render such methods clinically and commercially viable.

### SUMMARY OF THE INVENTION

The inventors have discovered compositions, methods, and devices that enable generation and expansion of memory like NK cells in a conceptually simple and efficient manner. Advantageously, memory like NK cells can be generated in a 2-step process in which NK cells are expanded to a desired quantity and in which the expanded NK cells are then induced with a mixture of cytokines to so form the cytokine induced memory like (CIML) NK cells. Expansion of the NK cells is preferably performed in an enrichment process that uses N-803 and an anti-CD16 agonist antibody and optionally an anti-CD3 antibody. Activation to obtain the memory like character is then performed with a combination of stimulatory cytokines, most preferably with IL-12/IL-15/IL-18 or an IL-18/IL-12-TxM fusion protein complex.

Unexpectedly, and besides upregulation of activation markers and IFN-γ secretion, so activated expanded memory like NK cells had an increased expression of CD25 and the NK activation receptor DNAM-1 and a downregulated expression of the inhibitory receptor, TIGIT, which presumably contributed or even caused the observed heightened toxicity against of the CIML NK cells. Most notably, the CIML NK cells presented herein even exhibited significant cytotoxicity against the otherwise NK resistant tumor cell line MS-1 at a relatively low effector to target ratio.

In one aspect of the inventive subject matter, the inventors contemplate a method of producing CIML NK cells with enhanced cytotoxicity that includes one step of isolating from a biological fluid a mixture of mononuclear cells, and another step of contacting the mixture of the mononuclear cells with an anti-CD16 antibody and N-803 to expand NK cells. In a further step, the expanded NK cells are contacted with a stimulatory cytokine composition (typically including an IL-18/IL-12-TxM fusion protein complex, a mixture of IL-12, N-803, and IL-18, or a mixture of IL-12, IL-15, and IL-18) to thereby generate the CIML NK cells with enhanced cytotoxicity. Where desired, contemplated methods may further comprise a step of contacting the CIML NK cells with N-803 after re-stimulating the CIML NK cells.

Preferably, but not necessarily, the biological fluid is whole blood or cord blood, and the mixture of mononuclear cells is not further processed to enrich NK cells. Most typically, the mixture of the mononuclear cells contains about 100-500×10⁶ cells, and/or the step of contacting the mixture is performed in a volume of between about 100-300 ml or at a cell density of about 1×10⁶ cells/ml. In further embodiments, the anti-CD16 antibody in the step of contacting the mixture may be present at a concentration of between 0.05-0.5 mcg/ml, and/or the N-803 in the step of contacting the mixture may be present at a concentration of between 0.1-1.0 nM. Optionally, the step of contacting the mixture may further include a step of contacting the mixture of the mononuclear cells with an anti-CD3 antibody (e.g., at an anti-CD3 antibody concentration of between 0.1-1.0 ng/ml).

While in some aspects the stimulatory cytokine composition includes the IL-18/IL-12-TxM fusion protein complex, in other aspects the stimulatory cytokine composition includes the mixture of IL-12, N-803, and IL-18, and in further aspects the stimulatory cytokine composition includes the mixture of IL-12, IL-15, and IL-18. Most typically, the NK cells are expanded to a total cell number of about 0.5-5.0×10⁹ cells, and/or the step of contacting the expanded NK cells with the stimulatory cytokine composition is performed in the same container as the step of expanding the NK cells.

Therefore, and viewed from a different perspective, the inventors also contemplate a method of activating NK cells to form CIML NK cells with enhanced cytotoxicity. Such methods will include a step of providing expanded NK cells (typically expanded from mononuclear cells of whole blood or cord blood) and a further step of contacting the expanded NK cells with a stimulatory cytokine composition that may include an IL-18/IL-12-TxM fusion protein complex, a mixture of IL-12, N-803, and IL-18, or a mixture of IL-12, IL-15, and IL-18 to so generate the CIML NK cells with enhanced cytotoxicity.

As noted before, it is contemplated that the NK cells are expanded from whole blood or from cord blood. Thus, the NK cells may be autologous relative to an individual receiving a transfusion comprising the CIML NK cells. Preferably, the stimulatory cytokine composition includes the IL-18/IL-12-TxM fusion protein complex. However, in further embodiments the stimulatory cytokine composition may also include a mixture of IL-12, N-803, and IL-18 or a mixture of IL-12, IL-15, and IL-18. Typically, the expanded NK cells have a total cell number of about 0.5-5.0×10⁹ cells.

It is further contemplated that the CIML NK cells with enhanced cytotoxicity will have cytotoxicity against MS-1 cells, that the CIML NK cells with enhanced cytotoxicity have a decreased expression of CD16 as compared to expanded NK cells that are contacted with N-803 alone, that the CIML NK cells with enhanced cytotoxicity have a decreased expression of TIGIT as compared to expanded NK cells that are contacted with N-803 alone, and/or that the CIML NK cells with enhanced cytotoxicity have an increased expression of CD25 and/or DNAM1 as compared to expanded NK cells that are contacted with N-803 alone.

Therefore, the inventors also contemplate a CIML NK cell with enhanced cytotoxicity that exhibits cytotoxicity against MS-1 cells of at least 50% killing at an effector to target cell ratio of equal or less than 5. In further aspects, the CIML NK cell has a decreased expression of CD16 as compared to expanded NK cells that are contacted with N-803 alone, has a decreased expression of TIGIT as compared to expanded NK cells that are contacted with N-803 alone, and/or has an increased expression of CD25 and/or DNAM1 as compared to expanded NK cells that are contacted with N-803 alone.

While not limiting the inventive subject matter, the CIML NK cell is preferably an autologous cell relative to an individual receiving a transfusion comprising the CIML NK cell. In other embodiments, the CIML NK cell may also be a recombinant NK cell. For example, such recombinant cells may express CD16 or a variant thereof, IL-2 or a variant thereof, and/or IL-15 or a variant thereof from a recombinant nucleic acid.

In still further contemplated aspects, the inventors also contemplate a pharmaceutical composition comprising a pharmaceutically acceptable carrier in combination with the CIML NK cells as presented herein. Consequently, use of the CIML NK cell as presented herein in medicine, and particularly in the treatment of cancer is contemplated.

Therefore, the inventors also contemplate a method of treating an individual with a CIML NK cell in need thereof that includes a step of administering to the individual a therapeutically effective amount of the CIML NK cell as presented herein. Preferably, the CIML NK cell is an autologous cell of the individual, and/or the CIML NK cell is a peripheral blood or cord blood derived NK cell.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts an exemplary schematic of an IL-18/IL-12-TxM fusion protein complex.

FIG. 2 depicts exemplary results from peripheral blood NK cell expansion using autologous PBMCs and selected combinations of specific antibodies.

FIG. 3 depicts exemplary results from a cytotoxicity assay of cord blood derived CIML NK cells against MS-1 target cells.

FIG. 4 depicts exemplary results for expression of selected phenotype markers on cord blood derived CIML NK cells.

FIG. 5 depicts exemplary results from a cytotoxicity assay of peripheral blood derived CIML NK cells against MS-1 target cells.

FIG. 6 depicts exemplary results for expression of selected phenotype markers on peripheral blood derived CIML NK cells.

FIG. 7 depicts an exemplary activation cluster phenotype of cord blood derived CIML NK cells after IL-18/12 TxM exposure.

FIG. 8 depicts an exemplary CD25 expression on cord blood derived CIML NK cells after IL-18/12 TxM exposure.

FIG. 9 depicts an exemplary activation cluster phenotype of cord blood derived CIML NK cells upon restimulation.

FIG. 10A-10C depict exemplary results for cell killing activity of cord blood derived CIML NK cells after 24 hours (10A), 48 hours (10B), and 72 hours (10C) IL-18/12 TxM exposure.

FIG. 11 depicts exemplary results for cell killing activity of cord blood derived CIML NK cells cultivated in a single-box culture environment and activation clustering from such cells.

FIG. 12 depicts exemplary results for NK marker expression on peripheral blood derived CIML NK cells after IL-18/12 TxM exposure.

FIG. 13 depicts exemplary results from a cytotoxicity assay of peripheral blood derived CIML NK cells after IL-18/12 TxM exposure against K562 target cells.

FIG. 14 depicts exemplary results for IFN-γ staining of peripheral blood derived CIML NK cells after re-stimulation.

FIG. 15 depicts exemplary results from a cytotoxicity assay of peripheral blood derived CIML NK cells after exposure to N-803.

FIG. 16 depicts exemplary results for IFN-γ staining of cord blood derived CIML NK cells after re-stimulation and exemplary results from a cytotoxicity assay after exposure to N-803.

### DETAILED DESCRIPTION

Immune therapies in the treatment of cancer increasingly make use of various cell-based components, and more recently NK cells have become a promising modality. While some NK cells are now available at relatively high quantities, production of therapeutically meaningful quantities of autologous NK cells and/or memory like NK cells have remained problematic at best. Unfortunately, many of the current methods require use of feeder layers or differentiation of isolated CD34+ hematopoietic stem cells (HSCs), which is both time and resource intensive. Moreover, due to the various manipulation steps needed, such methods typically require human interaction and are prone to contamination. In addition, conversion of NK cells to a memory like phenotype may reduce cytotoxicity in at least some protocols or may not deliver sufficient amounts of such cells.

The inventors have now discovered various systems, compositions, and methods to generate therapeutically meaningful quantities (e.g., at least 0.5×10⁹ NK cells) of NK cells that can be readily converted to memory like NK cells in a simple and effective manner that can even be fully automated once the mononuclear cells are obtained from a biological fluid (e.g., whole blood, cord blood). Advantageously, such NK cells can be autologous NK cells and can be induced to a memory like phenotype to yield cytokine induced memory like (CIML) NK cells with enhanced cytotoxicity. Notably, and as is described in more detail below, the so generated CIML NK cells will have superior cytotoxicity as compared to other (CIML) NK cells and even have significant killing capacity against target cells that are otherwise resistant or even inert to NK cell cytotoxicity such as MS-1 cells (Merkel cell carcinoma cells).

While not wishing to be bound by any theory or hypothesis, the inventors contemplate that the enhanced cytotoxicity may be due to the source of (naïve) NK cells, prior expansion conditions, and possibly to the uninterrupted (e.g., change in media, culture conditions, etc.) nature of expansion and cytokine induction, which may result in the over-expression of activating factors and the under-expression of inhibitory receptors. Among other notable features of the CIML NK cells presented herein, the CIML NK cells will typically exhibit cytotoxicity against MS-1 cells of at least 50% killing at an effector to target cell ratio of equal or less than 5, will have a decreased expression of TIGIT (inhibitory receptor) as compared to expanded NK cells that are contacted with N-803 alone, and an increased expression of CD25 and/or DNAM1 (activating co-receptor) as compared to expanded NK cells that are contacted with N-803 alone. Thus, the term “NK cells with enhanced cytotoxicity” refers to NK cells that exhibit cytotoxicity against MS-1 cells of at least 50% killing at an effector to target cell ratio of equal or less than 5, a decreased expression of TIGIT (inhibitory receptor) as compared to expanded NK cells that are contacted with N-803 alone, and/or an increased expression of CD25 and/or DNAM1 (activating co-receptor) as compared to expanded NK cells that are contacted with N-803 alone. Moreover, contemplated CIML NK cells will typically also exhibit a decreased expression of CD16. Most typically, the CIML NK cells will exhibit all three of the above parameters (i.e., cytotoxicity against otherwise NK resistant cells, increased expression of activating receptors, decreased expression of inhibitory receptors).

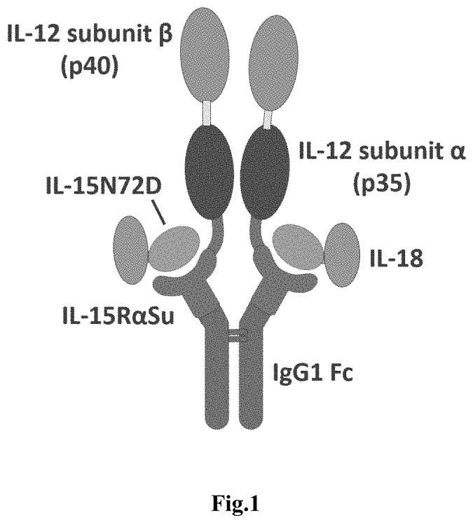

In one exemplary process contemplated herein, NK cells are in a first step expanded from a fraction of a biological fluid containing mononuclear cells, preferably to a total cell number of about 0.5-5.0×10⁹ cells. Notably, such expansion can be performed in a single reactor in a relatively small volume to a moderate cell density (e.g., 100-300 ml or at a cell density of about 0.5-5.0×10⁶ cells/ml) without the need for feeder cells or other manipulations that would require change of a culture vessel. Once a desired NK cell quantity is achieved, the so expanded NK cells are then in a second step contacted with a stimulatory cytokine composition to activate the NK cells to a memory like phenotype. Preferably, but not necessarily, the stimulatory cytokine composition will include an IL-18/IL-12-TxM fusion protein complex as is exemplarily depicted in FIG. 1. However, the stimulatory cytokine composition may also include a mixture of IL-12, N-803, and IL-18, or a mixture of IL-12, IL-15, and IL-18. Cytokine stimulation will typically be performed for a period of between 4-24 hours, and the so generated CIML NK cells may be rested or re-stimulated (preferably in the presence of N-803) prior to transfusion.

For example, whole blood or cord blood can employed as a starting material that is processed to obtain mononuclear cells. Most typically, processing can be done using conventional density gradient centrifugation (e.g., using Ficoll-Paque Plus™ (a hydrophilic soluble polysaccharide, density 1.077 g/mL), commercially available from GE Lifesciences). Once the mononuclear cells are separated from the centrifuge tube, the cells are washed and re-suspended in an activation medium (e.g., NK MACS supplemented with 10% human AB serum). The activation medium can further comprise N-803 at a concentration of about 0.4 nM, and an anti-CD16 antibody at a concentration of about 1.0 mcg/ml.

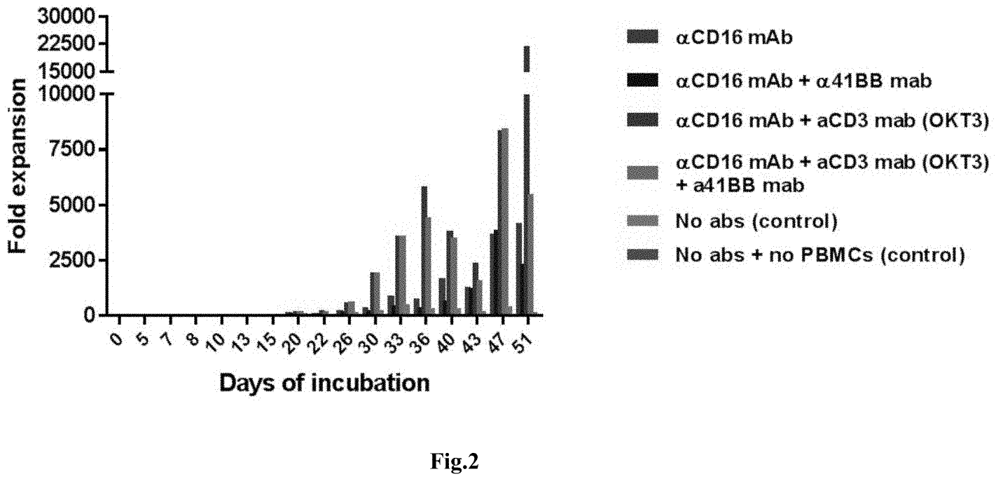

Most typically, the mononuclear cells have a density of 1-2×10⁶ cells/ml in a total volume of about 200 ml, and the cells and medium are in a single container. After about 3-4 days, the cells are fed with fresh medium containing N-803, and further feed cycles are performed about every three days through recovery, rapid expansion, and culture culmination. Notably, successful NK cell expansion in such scheme was significantly dependent on the proper choice of stimulatory factors as is exemplarily shown in FIG. 2. Here, a dramatic expansion of over 20,000-fold from day 0 was observed when using anti-CD3 and anti-CD16 monoclonal antibodies whereas anti-CD16 antibodies alone failed to produce the same dramatic effect. Notably, the presence of an anti-4-1BB antibody seemed to prematurely exhaust the proliferation of NK cells.

Cells culture is then terminated upon reaching a desired quantity, typically about 0.5-5.0×10⁹ total cells and/or upon reaching a desired expansion (e.g. at least 100-fold expansion). Notably, despite the apparent simplicity, the so obtained cell culture contains after about three weeks more than about 85% NK cells, with less than about 8% NKT cells, and with less than about 2.5% T cells, and less than about 1.2% double negative (DN) T cells. Moreover, it should be recognized that the entire culture process may be performed in a single container within a self-contained bioreactor, which substantially reduces risk of contamination and eliminates reagent and cell handling during the cultivation step.

Upon reaching a desired cell quantity, the cells can be transferred into a fresh medium for subsequent cytokine stimulation. Alternatively, the cytokine stimulation to generate the memory like phonotype can be performed in the same medium, typically by adding further medium with a stimulatory cytokine composition that includes an IL-18/IL-12-TxM fusion protein complex (or a mixture of IL-12, N-803, and IL-18, or a mixture of IL-12, IL-15, and IL-18). In most cases, the cytokine stimulation will be performed for a time of between about 4-24 hours, and more typically between 12-16 hours. As will be readily appreciated, the cells can then be transferred into a transfusion medium prior to transfusion. In addition, the phenotype and/or cytotoxicity of the CIML NK cells may be determined and exemplary results are shown in more detail below.

With respect to suitable biological fluids, it is generally contemplated that the fluids can be autologous relative to the individual that will receive the NK cells isolated in the methods presented herein. Therefore, especially preferred biological fluids include fresh whole blood, cord blood (frozen or fresh), and cells separated in a leukapheresis procedure. However, it should be appreciated that the biological fluid may also be any fluid that contains NK cells (typically among other cell types). For example, suitable alternative biological fluids include whole blood from allogenic donors, which may or may not be matched for a compatible MHC type. Therefore, samples in a blood bank that approach expiration date are deemed suitable for use, as well as freshly donated whole or stored cord blood by an individual other than the NK cell recipient. Moreover, it should be noted that where the biological fluid is the cord blood, the cord blood may be matched and donated after a sufficient MHC match with the NK cell recipient. Likewise, it should be noted that the manner of isolating or enriching mononuclear cells may vary considerably, and the person of ordinary skill in the art will be readily apprised of the most suitable methods of isolation and enrichment. For example, where the biological fluid is whole blood or cord blood, it is preferred that the fluid is processed via gradient density centrifugation using any suitable medium (e.g., Ficoll-Hypaque). Alternatively, mononuclear cells may be obtained directly from the patient by leukapheresis, or the biological fluid may be subjected to removal of red blood cells using antibodies. In still further methods, mononuclear cells may be isolated using magnetic bead separation where the beads are coated or otherwise coupled to antibodies binding the mononuclear cells.

Likewise, it should be recognized that the particular nature of the medium for activation and feeding need not be limited to NK MACS medium, but that all media known to support growth of NK cells are deemed suitable for use herein. Most preferably, however, defined media are used and may be supplemented with human AB serum.

Proliferation of the NK cells in the mixture of mononuclear cells is preferably stimulated and supported with a combination of an anti-CD16 antibody and N-803, and optionally an anti-CD3 antibody. There are various sources for anti-CD16 antibodies known in the art/commercially available, and particularly preferred anti-CD16 antibodies have agonist (activating) activity and are specific to human CD16. However, activators other than anti-CD16 antibodies are also deemed suitable for use herein include anti-CD16 antibody fragments and fusion proteins with anti-CD16 antibody fragments. Additionally, or alternatively, contemplated activators also include CD314 or NKG2D, the natural cytotoxicity receptors CD335 (NKp46), CD336 (NKp44) and CD337 (NKp30), CD226 (DNAM-1), CD244 (2B4), members of the CD158 or killer immunoglobulin-like receptor (KIR) family that carry a short cytoplasmic tail (KIR2DS and KIR3DS) and CD94/NKG2C, among others.

Concentrations of the anti-CD16 antibody will typically follow those already known in the art for activation of NK cells. Therefore, suitable concentrations for anti-CD16 antibodies will be between about 0.01-5.0 mcg/ml, and more typically between about 0.01-0.3 mcg/ml, or between about 0.05-0.5 mcg/ml, or between about 0.1-1.0 mcg/ml, or between about 1.0-5.0 mcg/ml. With respect to the duration of exposure to the anti-CD16 antibody it is generally contemplated that the mixture of mononuclear cells is exposed to only a single, two, or there doses of the anti-CD16 antibody, most typically when the mononuclear cells are isolated and contacted with the activation medium for the first (and/second, and/or third) time. The person of ordinary skill in the art will be readily able to recognize proper schedule and dosage to achieve NK cell activation. Most typically, exposure of the mononuclear cells to the anti-CD16 antibody is contemporaneous with exposure of the mononuclear cells with the N-803. However, in less preferred embodiments, exposure of the mononuclear cells to the anti-CD16 antibody is sequentially to exposure of the mononuclear cells with the N-803 (with exposure of the mononuclear cells to the anti-CD16 antibody first being the preferred sequence).

Where desired, proliferation stimulation/support may also include contacting the cells with anti-CD3 antibody, typically at the same time of contacting the cells with anti-CD16 antibody. As noted above, concentrations of the anti-CD3 antibody will typically follow those already known in the art for activation of NK cells. Therefore, suitable concentrations for anti-CD3 antibodies will be between about 0.01-10.0 ng/ml, and more typically between about 0.01-0.1 ng/ml, or between about 0.1-0.5 ng/ml, or between about 0.3-1.0 ng/ml, or between about 1.0-5.0 ng/ml. Likewise, with respect to the duration of exposure to the anti-CD3 antibody it is generally contemplated that the mixture of mononuclear cells is exposed to only a single, two, or there doses of the anti-CD3 antibody, most typically when the mononuclear cells are isolated and contacted with the activation medium for the first (and/second, and/or third) time. The person of ordinary skill in the art will be readily able to recognize proper schedule and dosage to achieve NK cell activation.

With respect to N-803 it is contemplated that N-803 (an IL-15N72D1L-15RαSu/IgG1 Fc complex with human sequences; see US 2019/0023766, commercially available from ImmunityBio) is preferred as an agent in the activation and feed medium. However, various alternative agents with IL-15 activity are also deemed suitable for use herein. In this context, and without wishing to be bound by any theory or hypothesis, the inventors contemplate that N-803 enables growth and expansion of the NK cells by virtue of continuous signaling. In contrast, IL-15 as isolated cytokine has a very short lifespan and signaling activity is typically very short. This, where IL-15 as isolated cytokine is added to a growth medium, the signaling will be pulsed or intermittently. In contrast, where N-803 is provided, stability of IL-15 is dramatically extended and signaling is deemed continuous. Moreover, it should be recognized that N-803 also provides a physiological context (i.e., IL-15 R-alpha chain) and a N72D form that acts as a super agonist. Therefore, any stabilized IL-15 compound is also expressly deemed suitable for use herein.

For example, all compounds and complexes that effect IL-15 signaling are deemed suitable for use herein so long as such compounds and complexes have a serum half-life that is longer than isolated/recombinant and purified IL-15 alone. Moreover, it is generally preferred that the stabilized IL-15 compounds will include at least portions of human sequences for IL-15 and/or IL-15 Rα. For example, suitable compounds include P22339 (a complex of IL-15 and the Sushi domain of IL-15Rα chain with a disulfide bond linking the IL-15/Sushi domain complex with an IgG1 Fc to augment its half-life; see Nature, Scientific Reports (2018) 8:7675), and XmAb24306, which is a IL-15/IL-15Rα-Fc heterodimer (see e.g., WO 2018/071919).

In further especially contemplated embodiments, the mixture of mononuclear cells is, after isolation from the biological fluid, placed into a cell culture container together with the medium containing the anti-CD16 (and optionally anti-CD3) antibody and N-803 to activate the NK cells. Most preferably, the container is a cell culture flask with at least one wall (or portion thereof) that is transparent to light such that cell shape, staining, and/or growth can be observed with a microscope or other optical instrument. Thus, it should be noted that the cells can be continuously or periodically monitored in a bioreactor, and so obtained measurements (e.g., cell size, cell number, cell distribution, etc.) can be used to trigger or modify an automated feeding schedule in a control unit that is logically coupled to the bioreactor. Most typically, and as shown in FIG. 2, feeding fresh medium with N-803 can be performed using a predefined schedule, typically every three days, where preferably each feeding will include N-803 to maintain continuous signaling. While the specific volumes in the examples below are suitable for expanding the NK cells to cell densities consistent with cell growth, it should be appreciated that the volumes may be adjusted to accommodate particular growth patterns. To that end, it should also be appreciated that the feeding may be continuously or that predetermined volumes may be changed in response to the growth kinetic observed in the container.

In most cases, the yield of the NK cells at the end of the cultivation will be typically at least 80%, or at least 82%, or at least 85%, or at least 88%, or at least 90%, or at least 92%, or at least 94% of all live cells with the remainder being NKT cells, DN T cells, and T cells. For example, remaining NKT cells will typically be equal or less than 10%, or equal or less than 8%, or equal or less than 7%, or equal or less than 6% of all live cells, while remaining T cells will typically be equal or less than 5%, or equal or less than 4%, or equal or less than 3%, or equal or less than 2% of all live cells, and remaining DN T cells will typically be equal or less than 3%, or equal or less than 2%, or equal or less than 1.5%, or equal or less than 1% of all live cells.

Therefore, and viewed from a different perspective, it should be appreciated that the systems and methods contemplated herein are capable of remarkably high expansion of NK cells, and typical expansions are at least 80-fold, or at least 100-fold, or at least 120-fold, or at least 130-fold, or at least 140-fold with respect to the number of NK cells originally present in the mixture of mononuclear cells. Such expansion is particularly notable in view of the very simple manner of activation and cultivating (one-pot process). Indeed, once the mixture of mononuclear cells is placed into the cell culture container, the entire process can continue within the same container and can be sustained by addition of media only. Thus, complex handling and expensive reagents are entirely avoided, and the risk for contamination is significantly reduced.

As already noted above, the NK cells can be expanded to a total cell number of about 0.1-1.0×10⁹ cells, or about 0.3-3.0×10⁹ cells, or about 0.5-5.0×10⁹ cells, or about 0.7-7.0×10⁹ cells, or about 1-10×10⁹ cells, or even higher. The exact number of expanded NK cells will typically depend, among other things, on the particular purpose for the NK cells, culture conditions, and the starting number of cells. Upon reaching the desired quantity of cells, cytokine stimulation may then be performed in the expansion medium, typically by adding fresh medium that contains a stimulatory cytokine composition.

In most cases, the stimulatory cytokine composition will comprise one or more activating cytokines such as IL-2, IL-12, IL-15, IL-21, and to a lesser degree also IL-4 and IL-7. Of course, and as discussed in more detail below, suitable cytokines may also be derivatives of the above cytokines, and especially preferred derivatives include fusion complexes. Still further, it should be recognized that one or more of the cytokines may also be expressed in the expanded NK cells following transfection with an appropriate recombinant nucleic acid (e.g., transient expression from a plasmid or viral expression vector).

For example in some embodiments, the stimulatory cytokine composition will comprise an IL-18/IL-12-TxM fusion protein complex, and especially preferred fusion protein complexes are described in WO 2018/165208, which is incorporated by reference herein. In such case, it should be appreciated that the fusion protein complex provides three cytokine functions (IL-12, IL-15, and IL-18) in a stabilized form via their coupling to an Fc portion of a human IgG. Moreover, while not wishing to be bound by any theory or hypothesis, the Fc portion of the fusion protein complex may provide a further stimulatory signal, possibly through interaction with CD16 on the expanded NK cells. However, other fusion protein complexes based on N-808 are also expressly contemplated herein. For example, suitable fusion protein complexes may include targeting scFv portions, or cytokine portions other than (or in addition to) IL-12 and IL-18. Of course, it should be noted that while an IL-18/IL-12-TxM fusion protein complex is in many cases preferred, alternative TxM fusion protein complexes are also deemed suitable and especially contemplated fusion complexes will include a IL15/IL-15Ralpha portion as described in WO 2018/165208, and at least one additional cytokine selected from the group consisting of IL-7, IL-18, and IL-21. Therefore, and among other suitable choices, contemplated TxM fusion complexes include an IL-18/IL-7 TxM and/or IL-18/IL-21 TxM.

Therefore, in other examples, the stimulatory cytokine composition may also comprise a derivative of IL-15, and especially preferred derivatives are those based on N-803. Such derivatives will advantageously have increased signaling effect as compared to IL-15 per se due to the presence of the IL-15Rα chain, and exemplary suitable derivatives are described in WO 2016/004060 and WO 2018/075989. Most typically, where N-803 or similar fusion proteins are used, additional cytokine functions will be supplied by individual cytokines, and especially IL-7, IL-12, IL-21, and IL-18. Therefore, in yet another aspect of the inventive subject matter, the stimulatory cytokine composition may also comprise IL-7, IL12, IL-15, IL-21, and IL-18 as individual cytokines. Therefore, and among other choices, such individual cytokines may be added alone or in combination with other individual cytokines or TxM constructs, each or which may be recombinant (or even recombinantly expressed in the cell).

Thus, it should be appreciated that one or more of the stimulatory cytokines can also be (temporarily) expressed from a recombinant nucleic acid that is transfected into the expanded NK cells. For example, suitable transfection methods include viral transfection where the recombinant nucleic acid is a viral expression vector. On the other hand, the recombinant nucleic acid may also be transfected into the cell using electroporation or lipofection using methods well known in the art. Furthermore, where electroporation or lipofection is employed, it is typically preferred that the nucleic acid is an RNA (however, DNA is also deemed suitable for use herein).

Regardless of the particular type of stimulatory cytokine composition, it is generally contemplated that the cytokine or cytokines are present in the medium at a concentration effective to generate a memory like phenotype of the NK cell. Therefore, suitable total cytokine concentrations will be between 0.1 nM and 1.0 nM, or between 0.5 nM and 5.0 nM, or between 1.0 nM and 10 nM, or between 10 nM and 50 nM, and in some cases even higher. Where multiple cytokines are used, it is generally preferred that the cytokines are present in substantially equimolar concentrations (+/−50% deviation). On the other hand, where the stimulatory cytokine composition comprises an IL-18/IL-12-TxM fusion protein complex, the complex may be present between 0.5 nM and 5.0 nM, or between 1.0 nM and 10 nM, or between 10 nM and 50 nM, or even higher.

With respect to the timing of the stimulatory cytokine composition it is generally preferred that the NK cells are first expanded to a desired (typically final) quantity prior to exposure to the stimulatory cytokine composition. However, in alternative aspects, the stimulatory cytokine composition can be added to the expanding NK cell population starting at about 70% of the final desired cell quantity, or starting at about 80% of the final desired cell quantity, or starting at about 90% of the final desired cell quantity. In most aspects of the inventive subject matter, the exposure to the stimulatory composition will last be between about 2 hours and 48 hours, or between 4 hours and 8 hours, or between 8 hours and 12 hours, or between 12 hours and 24 hours, and in some cases even longer.

Exposure to the stimulatory cytokine composition can be terminated by replacement of the medium, typically with fresh medium or a medium suitable for transfusion. On the other hand, it is also contemplated that the so generated CIML NK cells can be subjected to a resting period prior to subsequent use that can last that between 0-4 hours, between 4-12 hours, between 12 and 24 hours, or between 1-4 days, and even longer. As will also be readily appreciated, the CIML NK cells may also be subjected to re-stimulation to further increase cytotoxicity, and re-stimulation will typically be performed using at least one stimulatory cytokine such as IL2 or IL-15. Most preferably, and as is shown in more detail below, re-stimulation provided unexpectedly high cytotoxicity where N-803 was used (as compared to IL-15 per se). Moreover, it should be noted that re-stimulation will typically follow standard protocols well known in the art.

Regardless of the final treatment of the CIML NK cells, it is contemplated that the CIML NK cells will be used for transfusion to an individual in need thereof, and most typically, the individual will be diagnosed with a cancer. As will also be readily appreciated, the CIML NK cells may form of a treatment regimen in which the individual receives a cancer vaccine (e.g., recombinant (adeno)viral vaccine, recombinant yeast vaccine, recombinant bacterial vaccine), a chemotherapeutic agent, a checkpoint inhibitor, N-803 or a TxM-based therapeutic, and/or a targeted interleukin (e.g., NHS-IL12).

While not limiting to the inventive subject matter, it is further contemplated that the CIML NK cells are expanded and/or activated in a culture environment that allows for continuous monitoring, continuous management of CO₂ and O₂ levels, and continuous monitoring to detect cell density (e.g., confluence). Among other options for such environments, especially preferred environments are automated cell culturing and harvesting devices as are described, for example, in WO 2015/165700. Such ‘GMB-in-a-box’ systems beneficially allow control over feeding schedules, gas control, allow for real-time detection of cell density, growth (kinetics) and cell health, as well as dramatically reduce the possibility of contamination due to significantly reduced handling requirements.

In still further contemplated aspects, it should be noted that the systems and methods presented herein advantageously also allow generation of CD56^{dim} and CD56^{bright} NK cells, particularly where the NK cells are generated from peripheral blood. Depending on further culture conditions, CD56^{bright} NK cells may then differentiate to CD56^{dim} cells. Such distinct NK cell populations can then be employed as for distinct therapeutic options due to their distinct maturation and cytotoxicity profile. Additionally, it should be appreciated that the compositions, systems and methods will also be suitable to generate NKT cells upon proper stimulation and culture.

### Examples

In view of the above, and as provided in more detail below, one exemplary method entailed isolating CBMCs or PBMCs by a single Ficoll centrifugation step, which was followed by incubation of the cells with about 0.4 nM N-803 and about 0.1 mcg/ml of an anti-CD16 antibody (e.g., clone B73.1, commercially available from BD Biosciences), and optionally about 0.5 ng/ml of an anti-CD3 antibody in NK MACS media with 10% human AB serum. Typically 100-150 mL (typically 135 mL) of CBMCs at a million cells/ml were used as the starting material with above reagents. Media was used for dilution with N-803 twice a week (3-5 day intervals) with a regimen of a 1:2 and 1:10 compared to existing volume with corresponding concentration of N-803 for a final concentration of 0.4 nM. The expansion culture is typically terminated when the expanded NK cells make up about between 90% and 99% (e.g., 98%) of all cells. Upon termination, cytokine induction can be performed as described in more detail below.

MNCs were freshly isolated from cord blood or peripheral blood. It was washed twice with complete NKMACS medium (NKMACS+Supplements+10% hu-AB-serum). MNCs were suspended in 150 mL of medium with density of 1×10\hat{ }6 cell/mL in a GMP box (500 mL volume). 150 mL cell suspension was supplemented with anti-CD16 antibody (1 mcg/mL) and N-803 (0.4 nM). GMP Box started imaging and cells were propagated according to pre-programmed steps. Cells in the GMP box were supplemented with 10× cytokine medium or with 2× cytokine medium in alternate fashion. NK enrichment (phenotype for CD3, CD56, and CD16 expression) and cell health (cell number, viability, and cell density) were monitored regularly and plotted.

Cytokine induction to generate CIML NK cells from expanded NK cells was started upon reaching a point at which 98% of all cells were NK cells. To that end, a box with 500 mL and 2.3×10\hat{ }6 cells/mL density was equally split into two separate boxes. Thus, 500 mL cell suspension became 250 mL in two the respective boxes and the cells were diluted 1:1 with fresh medium. Subsequently, IL18/12 TxM was added to a final concentration of 10 nM (for control and comparison, N-803 was used at a final concentration of 0.07 nM) and the cells were incubated with the IL-18/IL-12-TxM fusion protein complex for 16 hours to so obtain the CIML NK cells. For further testing, the cells were washed and then subjected to expression analyses and cytotoxicity assays.

Materials: MNCs from Cord and Peripheral Blood, anti-CD16 antibody, BD bioscience San Diego Calif.; NK MACS medium with NK supplement, staining antibodies for phenotyping (aCD3, aCD16, aCD56, aNKp30, aNKp44, aNKp46, aNKG2A, aNKG2D, aTIGIT, aCD34, aTRAIL, aCD57, aCXCR3, and aCCR5), Miltenyi Biotec San Diego, Calif.; Human AB serum, Access Biologicals, San Diego Calif.; N-803, GMP in a Box kit, Nantbio Inc. Culver City Calif. IL-18/IL-12-TxM fusion protein complex was obtained from ImmunityBio.

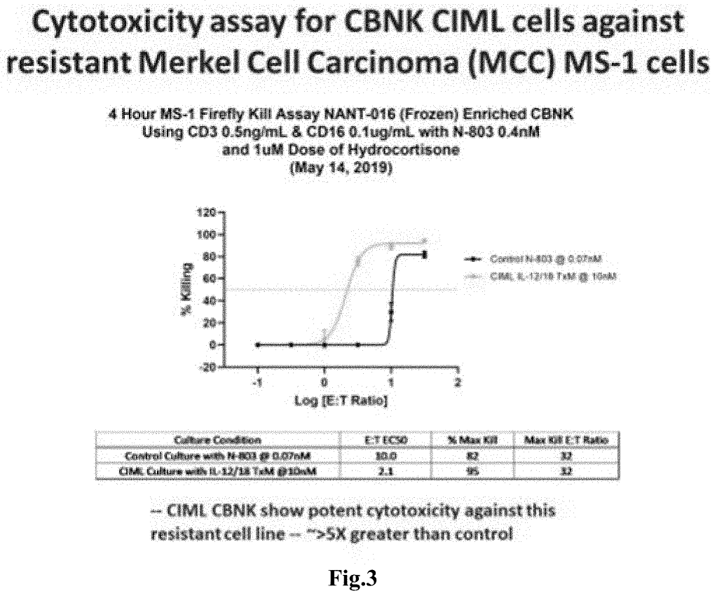

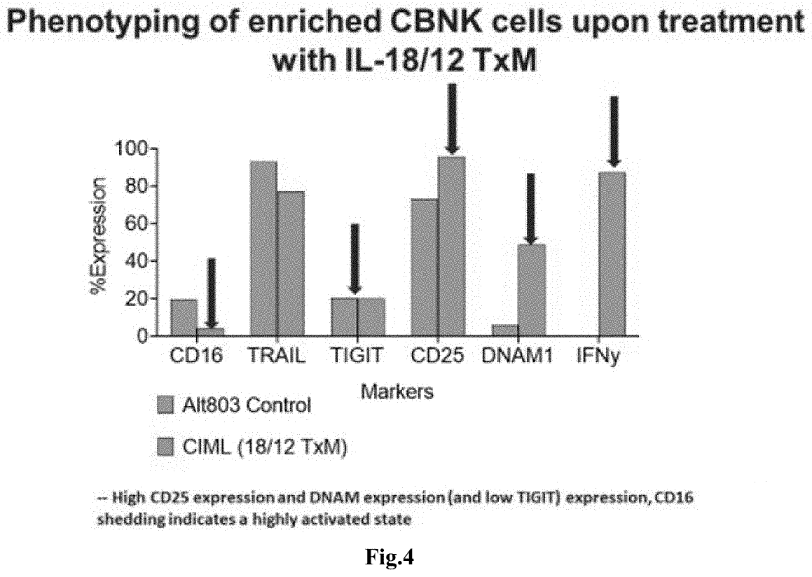

So generated CIML NK cells were tested for cytotoxicity and selected surface marker expression. More particularly, in one set of experiments cord blood derived CIML NK cells were tested against Merkel cell carcinoma cells (here MS-1 cells) that are typically resistant to NK cytotoxicity. Notably, and as can be seen in FIG. 3, the CIML NK cells had significant cytotoxicity after expansion and control exposure to the IL-18/IL-12-TxM fusion protein complex, whereas some cytotoxicity was even observed when the cord blood cells were exposed to N-803 only. FIG. 4 depicts exemplary results for surface marker expression in cord blood derived cells exposed to the IL-18/IL-12-TxM fusion protein complex and N-803. As can be seen, the CIML NK cells had reduced expression of CD16, but substantially increased expression of CD25, DNAM1, and strong secretion of IFN-γ.

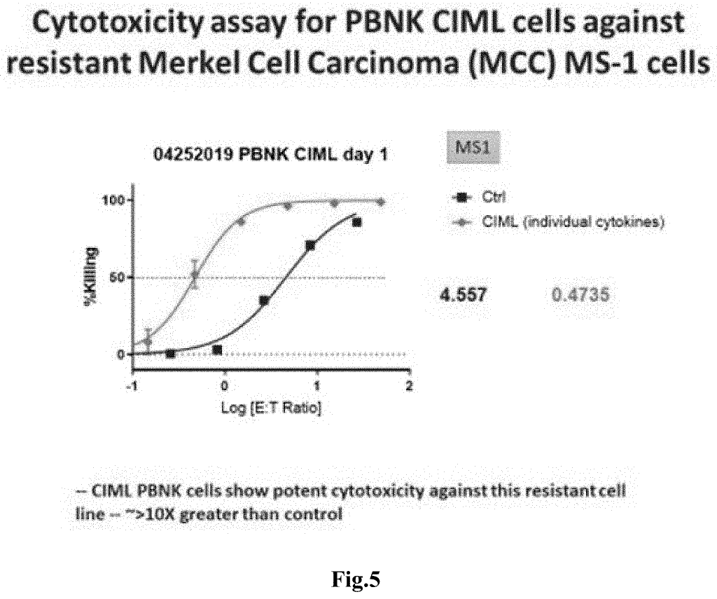

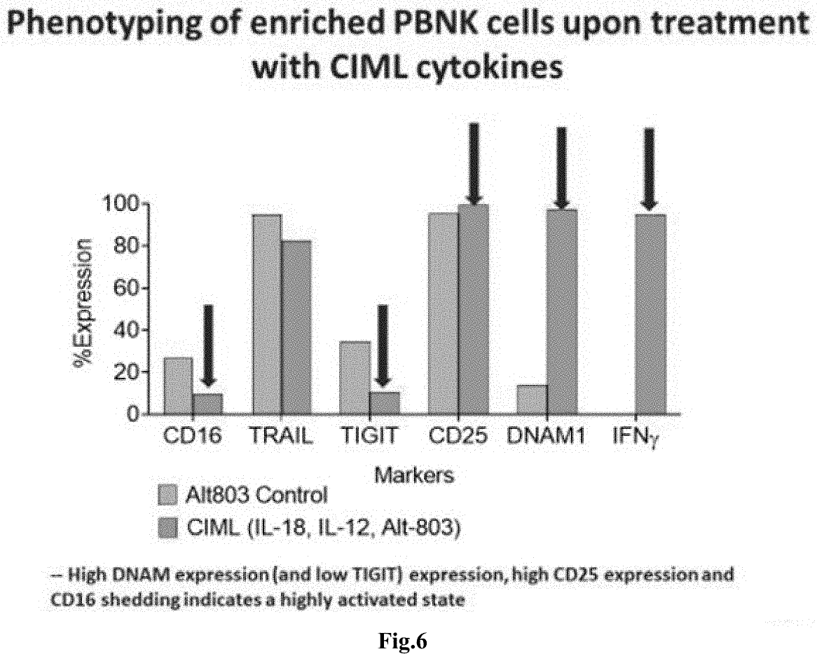

Similar results were obtained when the CIML NK cells were derived from peripheral blood as can be seen in FIG. 5. Here, CIML NK cells had substantial cytotoxicity against the MS-1 cell line, and the N-803 control cells from peripheral blood also showed some cytotoxicity. Likewise, the surface markers for the peripheral blood derived CIML NK cells showed decreased expression of CD16 and TIGIT, while having significant increases in CD25, DNAM1, and IFN-γ secretion as can be taken from FIG. 6. Notably, when NK cells were cultivated using standard cultivation protocols or where fresh NK cells were used, no significant cytotoxicity against MS-1 cells were observed, even where the cells were induced with IL-12, IL-15, and IL-18 to trigger a memory like phenotype.

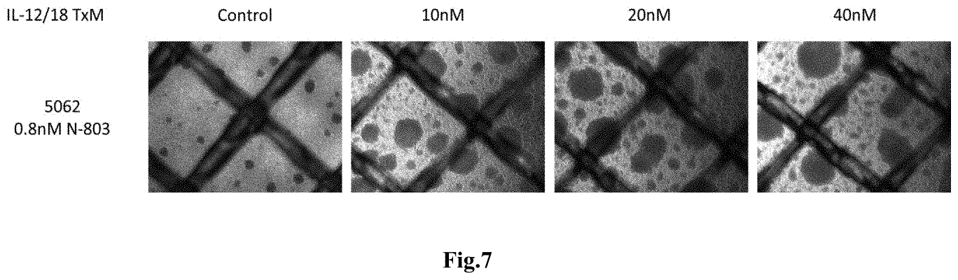

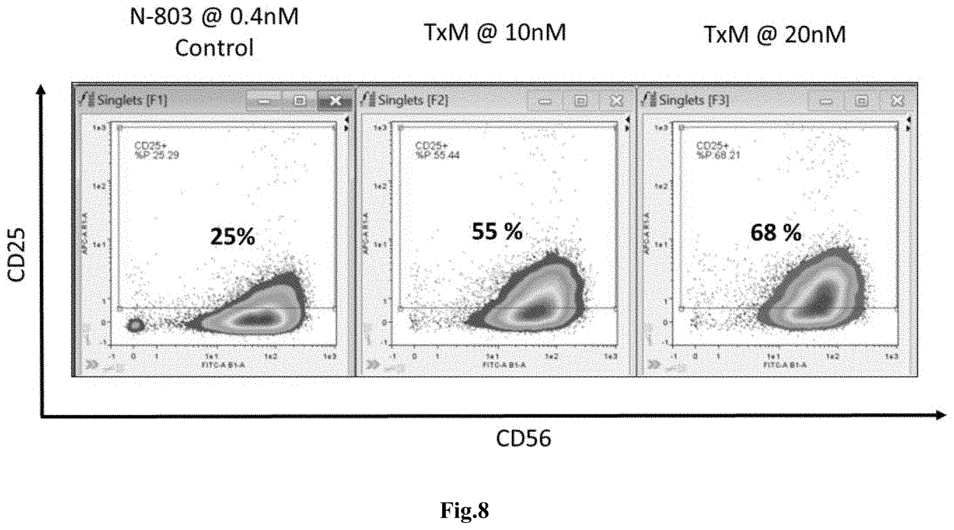

Cord blood derived CIML NK cells were also tested for the activation cluster phenotype and FIG. 7 depicts exemplary results comparing control exposure with N-803 with exposure to the IL-18/IL-12-TxM fusion protein complex. As can be seen from the images, there is a striking difference in the culture morphology after overnight exposure to the IL-18/IL-12-TxM fusion protein complex versus exposure to N-803. When looking at selected surface markers of these CIML NK cells, it was yet again apparent that exposure to the IL-18/IL-12-TxM fusion protein complex resulted in a significant increase of CD25 (which is a known activation associated receptor) as shown in FIG. 8. Clearly, cytokine stimulation with IL-12, IL-15, IL-18 functions substantially increased the CD25 presentation, which is typically not observed (at least to that degree) with conventional fresh NK cells.

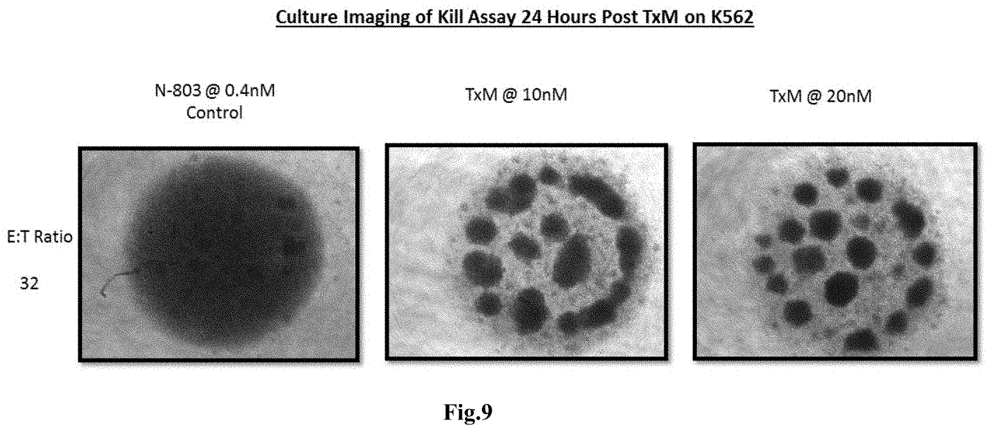

Such increase in activating receptors and decrease in inhibitory receptors was also readily evident when observing culture morphology in a kill assay on K562 cells as is shown in FIG. 9. Here, the cord blood derived CIML cells upon re-stimulation exhibited substantially increased activation clustering as compared to incubation with N-803.

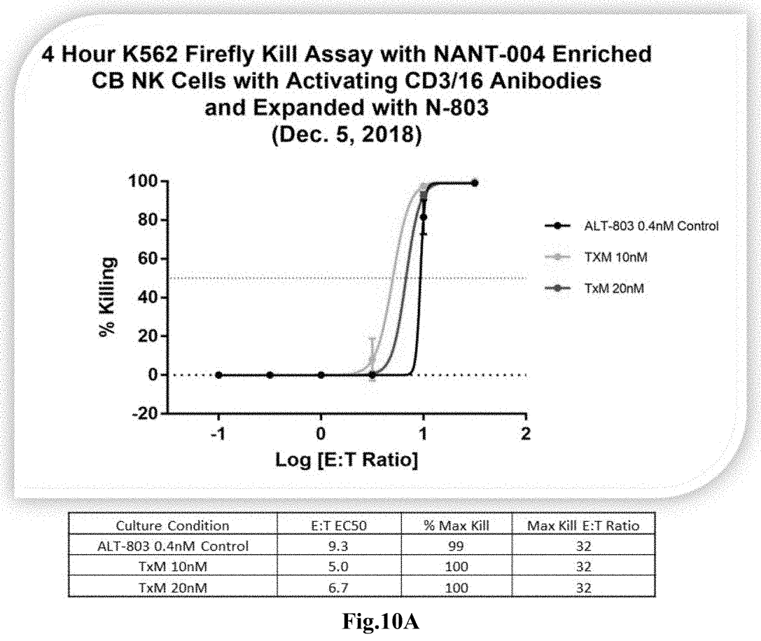

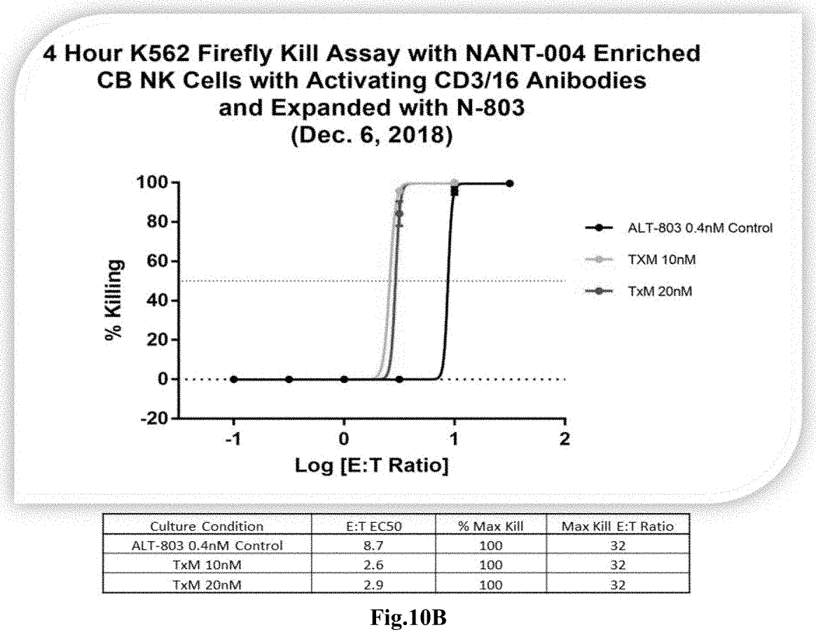

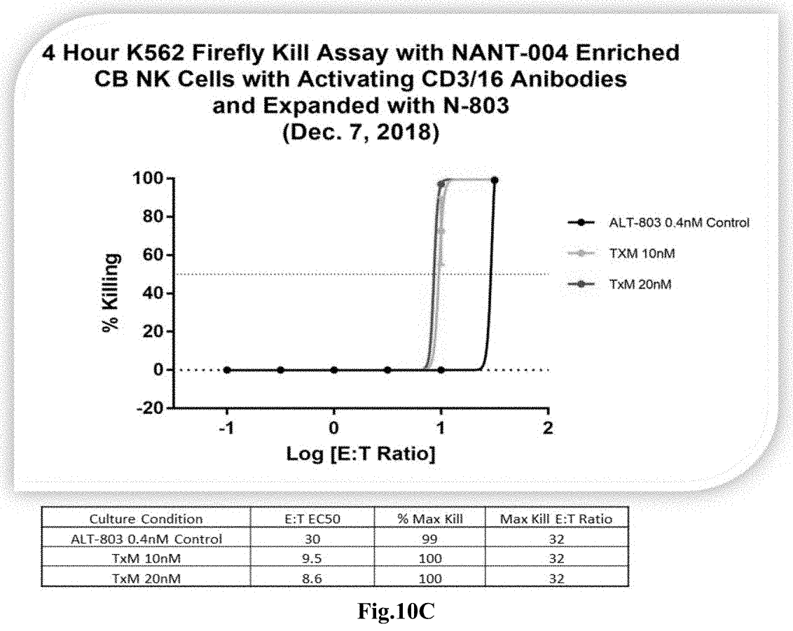

In further experiments, the inventors also investigated the time course of cytotoxicity on K562 cells as is exemplarily depicted in FIG. 10A, FIG. 10B, and FIG. 10C showing results after 24 hours, 48 hours, and 72 hours, respectively. After the first 24 hour time-point (FIG. 10A) one can see the start of increased killing capacity on K562 cells as both TxM concentrations tested have lower EC₅₀s than the N-803 control. At 48 hours (FIG. 10B) increased killing by the TxM treated samples is seen, but less so for the N-803 control. At this time-point the increase in killing of K562 is about 3-fold. At 72 hours (FIG. 10C) all conditions have begun to lose activity on K562 killing, but the TxM treated cells retain their enhanced killing by about 3-fold compared to the control.

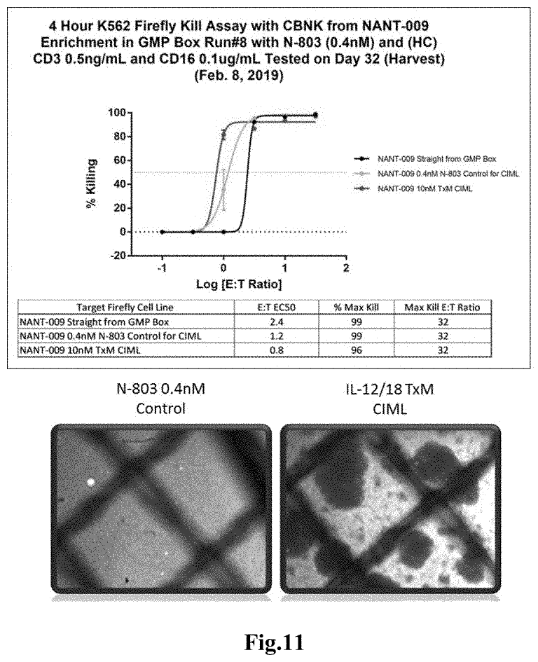

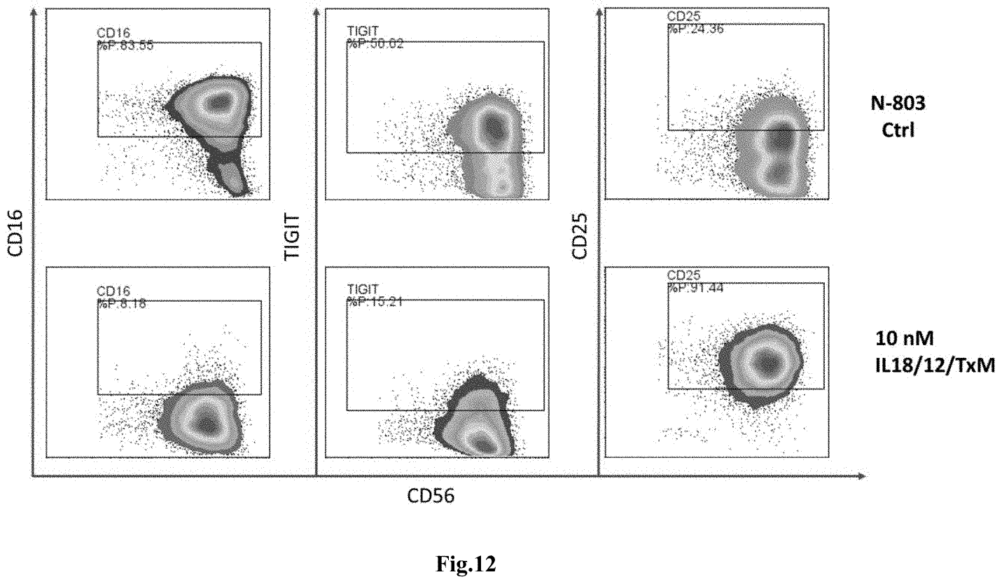

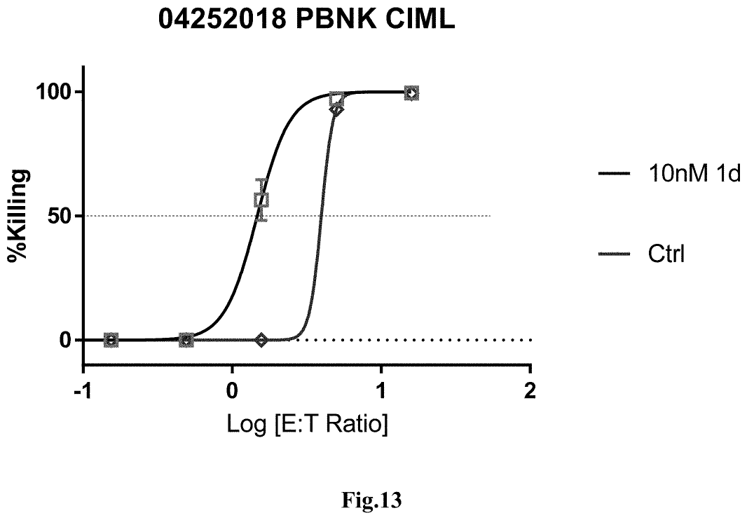

FIG. 11 provides a direct comparison for expanded cord blood derived NK cells, expanded cord blood derived NK cells with N-803 stimulation, and expanded cord blood derived NK cells stimulated with the IL-18/IL-12-TxM fusion protein complex for 24 hours. As can be seen, the cytotoxicity for all cells is readily evident, with expanded NK cells having a slight advantage over % max kill, but requiring a substantially higher E;T ratio as compared to the CIML cells. FIG. 12 depicts expression of selected markers of expanded peripheral blood derived NK cells with N-803 stimulation versus expanded peripheral blood derived NK cells stimulated with the IL-18/IL-12-TxM fusion protein complex. As can be taken from FIG. 12, there is a significant downregulation of TIGIT (and CD16) and a significant upregulation of CD25, which is indicative of activation. It should be noted that the downregulation of CD16 may be accompanied by a reduction in ADCC. However, the potential reduction in ADCC is outbalanced by the higher activation and cytotoxicity against cell lines that would otherwise be resistant to NK cell cytotoxicity. Similar cytotoxicity results are found with peripheral blood derived CIML NK cells after 24 hours stimulation with an IL-18/IL-12-TxM fusion protein complex as is shown in FIG. 13. Clearly, exposure to 10 nM of the IL-18/IL-12-TxM fusion protein complex generated better cell killing in the K562 assay.

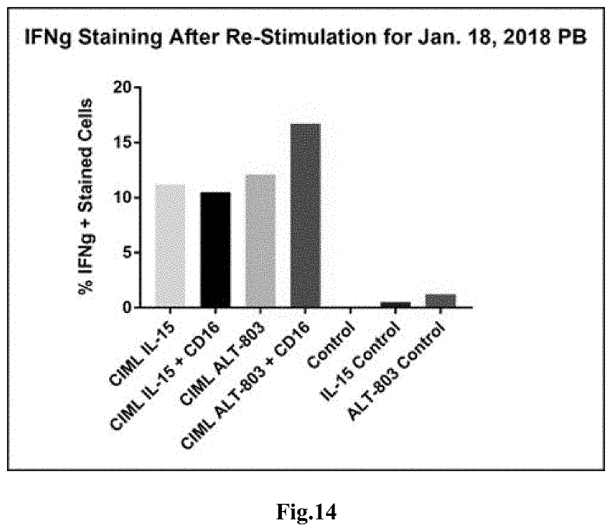

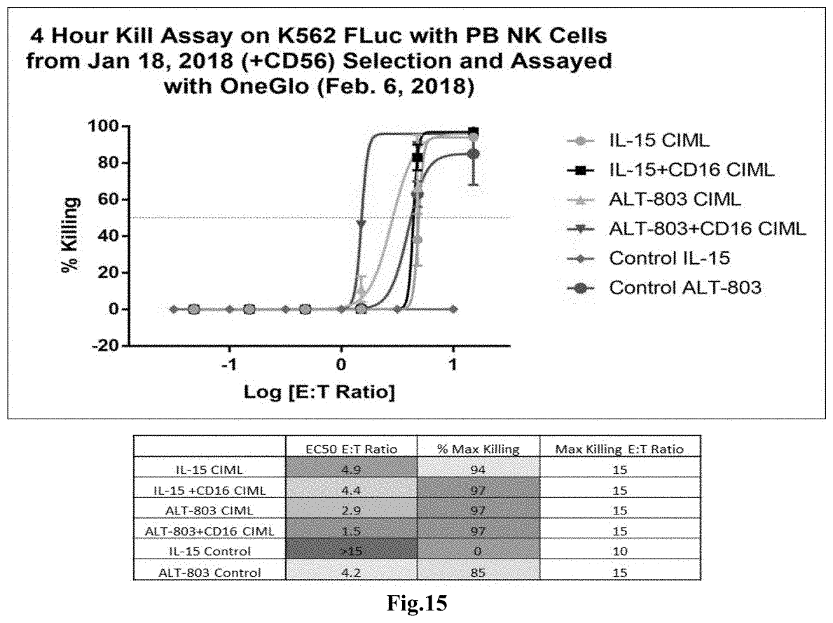

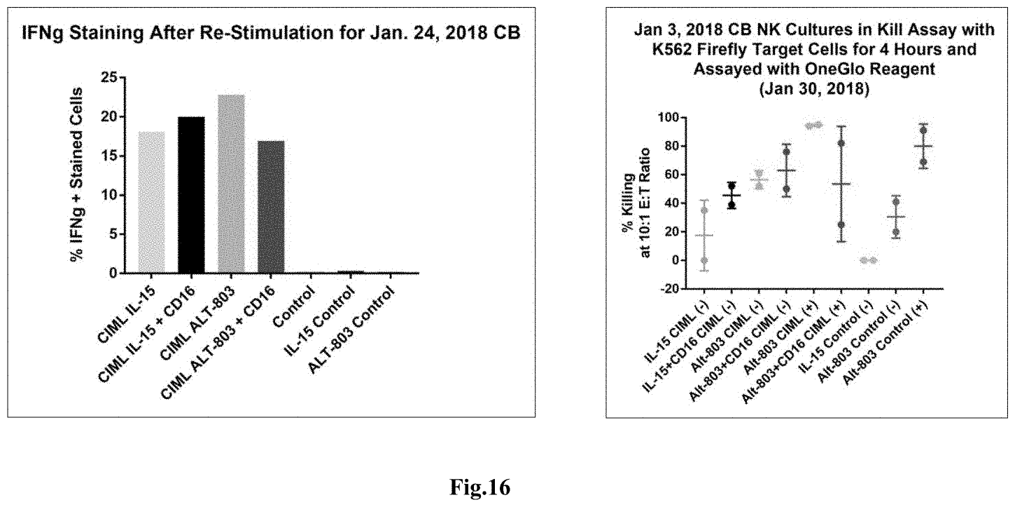

Secretion of IFN-γ was tested for peripheral blood derived CIML NK cells and FIG. 14 shows exemplary results using different conditions. The same cells were also used in a cytotoxicity assay and FIG. 15 shows exemplary results. Similar results are provided for cord blood derived CIML NK cells as can be taken from FIG. 16. Notably, cytokine induction with N-803 outperformed induction with IL-15 per se. Thus, it should be noted that while multi-cytokine induction is preferred as shown above, induction with N-803 is also expressly contemplated.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the cells or exosomes are administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient. Therefore, it should be appreciated that contemplated systems and methods can be considered a complete drug discovery system (e.g., drug discovery, treatment protocol, validation, etc.) for highly personalized cancer treatment.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1-15. (canceled)

16. A cord blood or whole blood derived cytokine induced memory like (CIML) NK cell with enhanced cytotoxicity exhibiting cytotoxicity against Merkel cell carcinoma (MS-1) cells of at least 50% killing at an effector to target cell ratio of equal or less than 5.

17. The CIML NK cell of claim 16 wherein the CIML NK cell has a decreased expression of CD16 as compared to expanded NK cells that are contacted with N-803 alone, or wherein the CIML NK cell has a decreased expression of TIGIT as compared to expanded NK cells that are contacted with N-803 alone.

18. The CIML NK cell of claim 16 wherein the CIML NK cell has an increased expression of CD25 and/or DNAM1 as compared to expanded NK cells that are contacted with N-803 alone.

19. The CIML NK cell of claim 16 wherein the CIML NK cell is an autologous cell relative to an individual receiving a transfusion comprising the CIML NK cell.

20. The CIML NK cell of claim 16 wherein the CIML NK cell is a recombinant NK cell.
